(19) United States
(12) Patent Application Publication
(10) Pub. No.: US 2005/0222683 A1
(43) Pub. Date: Oct. 6, 2005

(54) Shape memory alloy disc replacement device

(75) Inventor: Bret M. Berry, Jacksonville, FL (US)

Correspondence Address: HAYNES AND BOONE, LLP, 901 MAIN ST, SUITE 3100, DALLAS, TX, 75202, US

(73) Assignee: SDGI Holdings, Wilmington, DE (US)

(21) Appl. No.: 10/814,362

(22) Filed: Mar. 31, 2004

(51) Int. Cl. A61F 2/44

(52) U.S. Cl. 623/17.13

(57) ABSTRACT

A disc replacement device, such as for replacing a spinal disc between two vertebral bodies of the spine. The disc replacement device comprises an upper shell, a lower shell, and a plurality of compressible pillars each connecting the upper and lower shells and comprising a shape memory alloy. At least one of the plurality of pillars is interiorly offset from perimeters of the upper and lower shells. The disc replacement device may also include one or more fins comprising a shape memory alloy.

### BACKGROUND

The present disclosure relates generally to artificial replacement devices, and more particularly, to artificial disc replacement devices comprising shape memory alloys.

Intervertebral discs are located between the concave articular surfaces of the adjacent vertebral body endplates. They form an important and unique articulating system in the spine, allowing for multiplanar motion. In general, they permit movements such as flexion, extension, lateral flexion, and rotation.

Disc replacement devices have been used to repair and/or replace injured or damaged intervertebral discs. However, previous disc replacement devices possess a number of disadvantages. For example, to allow movement, some disc replacement devices contain mobile parts. To implant such a device, extensive disc space preparation is required, and the device may still protrude from the disc space. In addition, over time, the device may wear against the adjacent vertebral bodies and generate debris in the disc space. As a result, it may fail to function properly.

Other disc replacement devices have eliminated mobile parts by utilizing liquids or gels to produce motion. However, such liquids or gels must be properly contained, and their leakage may cause unwanted results.

Accordingly, what is needed in the art is a device, and methods of manufacture and use thereof, that address the above-discussed issues.

### SUMMARY

The present disclosure introduces a disc replacement device, such as for replacing a spinal disc between two vertebral bodies of the spine. One embodiment of a disc replacement device according to aspects of the present disclosure comprises an upper shell, a lower shell, and a plurality of compressible pillars each connecting the upper and lower shells. The pillars each comprise a shape memory alloy. At least one of the plurality of pillars is interiorly offset from perimeters of the upper and lower shells.

Another embodiment of a disc replacement device according to aspects of the present disclosure comprises upper and lower shells, one or more upper fins extending from a superior surface of the upper shell, and one or more lower fins extending from an exterior surface of the lower shell. At least one of the one or more upper fins comprises a first shape memory alloy, and at least one of the one or more lower fins comprises a second shape memory alloy. Such an embodiment may also include one or more pillars connecting the upper shell and the lower shell, wherein at least one of the one or more pillars comprises a third shape memory alloy. The first, second, and third memory alloys may be substantially different or similar.

A method of manufacturing a disc replacement device is also introduced in the present disclosure. In one embodiment, the method includes providing a shape memory alloy body. Thereafter, material is removed from the body to form a plurality of through-holes through the body, thereby defining from the body upper and lower shells and a plurality of integral pillars extending between the upper and lower shells.

The present disclosure also introduces a method of installing a disc replacement device. Such a method may include providing a disc replacement device having upper and lower shells and a plurality of compressible pillars extending between the upper and lower shells. The plurality of pillars each comprise a shape memory alloy, and at least one of the plurality of pillars is interiorly offset from perimeters of the upper and lowers shells. The installation method also includes positioning the disc replacement device between adjacent vertebral bodies.

The foregoing has outlined various features of several embodiments so that those skilled in the art may better understand the detailed description that follows. Additional features will be described below that further form the subject of the claims herein. Those skilled in the art should appreciate that they can readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure.

### BRIEF DESCRIPTION OF THE DRAWINGS

Aspects of the present disclosure are best understood from the following detailed description when read with the accompanying figures. It is emphasized that, in accordance with the standard practice in the industry, various features are not drawn to scale. In fact, the dimensions of the various features may be arbitrarily increased or reduced for clarity of discussion.

FIG. 1 is a perspective view of one embodiment of a disc replacement device according to aspects of the present disclosure.

FIG. 2 is a front view of the disc replacement device shown in FIG. 1.

FIG. 3 is a side view of the disc replacement device shown in FIG. 1.

FIG. 4 is a top view of the disc replacement device shown in FIG. 1.

FIG. 5 is a perspective view of another embodiment of a disc replacement device according to aspects of the present disclosure.

FIG. 6 is a front view of the disc replacement device shown in FIG. 5.

FIG. 7 is a perspective view of another embodiment of a disc replacement device according to aspects of the present disclosure.

FIG. 8 is a front view of the disc replacement device shown in FIG. 7.

FIG. 9 is a perspective view of another embodiment of a disc replacement device in a compressed configuration according to aspects of the present disclosure.

FIG. 10 is a front view of the disc replacement device shown in FIG. 9.

FIG. 11 is a side view of the disc replacement device shown in FIG. 9.

FIG. 12 is a top view of the disc replacement device shown in FIG. 9.

FIG. 13 is a perspective view of another embodiment of disc replacement devices constructed according to aspects of the present disclosure.

FIG. 14 is a front view of an embodiment of two disc replacement devices positioned in a disc space between two adjacent vertebral bodies according to aspects of the present disclosure.

FIG. 15 is a side view of the disc replacement devices shown in FIG. 14.

FIG. 16 is a perspective view of an embodiment of a method of insertion of a disc replacement device via an insertion device according to aspects of the present disclosure.

FIG. 17 is a perspective view of another embodiment of a method of insertion of a disc replacement device according to aspects of the present disclosure.

### DETAILED DESCRIPTION

It is to be understood that the following disclosure provides many different embodiments, or examples, for implementing different features of various embodiments. Specific examples of components and arrangements are described below to simplify the present disclosure. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed. Moreover, the formation of a first feature over or on a second feature in the description that follows may include embodiments in which the first and second features are formed in direct contact, and may also include embodiments in which additional features may be formed interposing the first and second features, such that the first and second features may not be in direct contact.

Embodiments constructed according to aspects of the present disclosure may utilize properties of shape memory alloys. A shape memory alloy may be deformed during its martensitic phase, but will regain its original shape when it is heated above a certain temperature, such as an austenite phase temperature. In addition, at above a certain temperature, certain shape memory alloys may exhibit a superelastic property, thereby able to absorb large deformations without damaging their structures. As a result, a device made of certain shape memory alloys may deform under a load or strain and recover its original shape after the load or strain is removed.

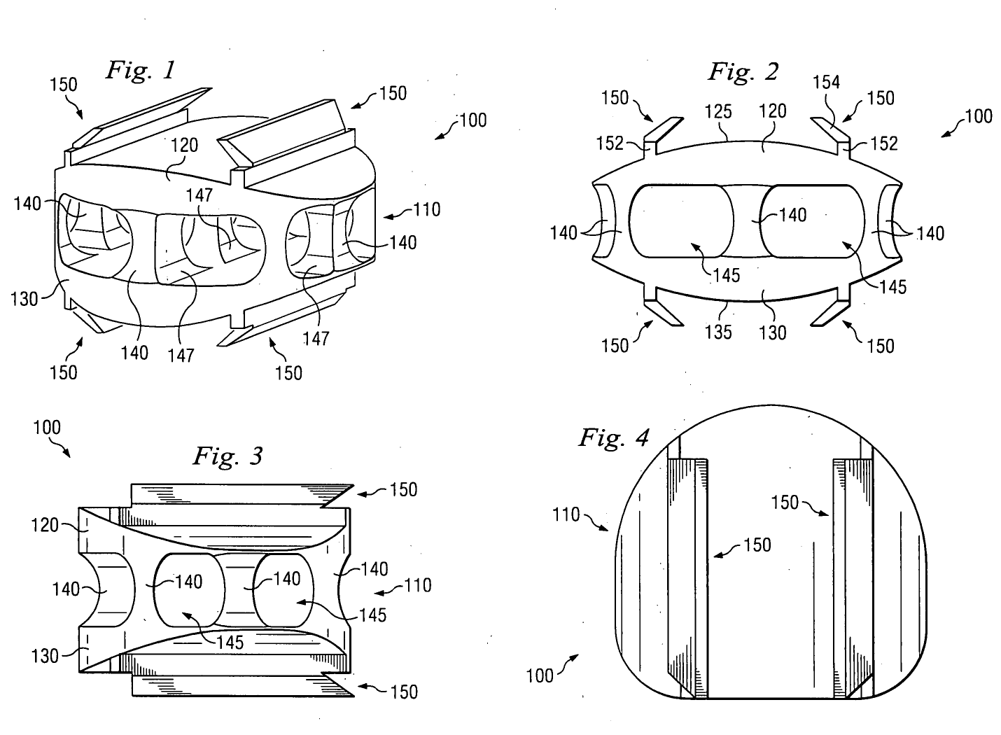

Referring to FIGS. 1-4 collectively, illustrated are various views of one embodiment of a disc replacement device 100 according to aspects of the present disclosure. The disc replacement device 100 may include a body 110 comprising an upper shell 120 and a lower shell 130. The upper and lower shells 120, 130 may be integral components, formed from a single bulk material, or may be separate components bonded or otherwise coupled together. The disc replacement device 100 may also include one or more pillars 140 extending between the upper and lower shells 120, 130. The pillars 140 may also be integral to one or both of the upper and lower shells 120, 130, or may be discrete components bonded or otherwise coupled to the upper and lower shells 120, 130. Some embodiments of the disc replacement device 100 may also include one or more fins 150. The fins 150 may also be formed integral to the upper and/or lower shells 120, 130, or may be discrete components bonded or otherwise coupled to the upper and/or lower shells 120, 130.

The disc replacement device 100 may comprise any biomedical-compatible structural material. For example, the upper and lower shells 120, 130 may comprise stainless steel, titanium, shape memory alloys, polymers, carbon fiber, porous materials, and/or other materials. The upper and lower shells 120, 130 may comprise substantially similar or identical compositions, or may have differing compositions.

The pillars 140 and the fins 150, when included, at least partially comprise one or more shape memory alloys. Thus, the pillars 140 and the fins 150 may each deform under a load or strain. Moreover, the pillars 140 and the fins 150 may be superelastic within certain temperature ranges, possibly within a temperature range of a live human body. For example, the pillars 140 and the fins 150 may be superelastic at a temperature of above about 34° C.

The pillars 140 and the fins 150 may also each comprise substantially similar or identical shape memory alloys. However, in other embodiments, the pillars 140 may comprise varying shape memory alloys or percentages thereof, and the fins 150 may comprise varying shape memory alloys or percentages thereof. In one embodiment, one or more of the pillars 140 and/or the fins 150 comprises Nitinol, which may include substantially equal portions of nickel and titanium. The Nitinol maybe treated in hot air and then ice water to produce an austenite temperature that is lower than the temperature range of a live human body. In one embodiment, the pillars 140 and the fins 150 may have an austenite temperature of approximately 34° C. Accordingly, at above 34° C., the pillars 140 and fins 150 may be superelastic. In a similar embodiment, one or more of the pillars 140 and/or the fins 150 may also or alternatively comprise a copper-based alloy.

As in the illustrated embodiment, the upper shell 120 and the lower shell 130 may be substantially identical. However, it is contemplated that without deviating from the spirit and scope of the present disclosure, the upper shell 120 and the lower shell 130 may have different shapes and/or compositions.

The upper and lower shells 120, 130 may be at least partially cylindrical and, thereby, may include contacting surfaces 125, 135, respectively, that are substantially cylindrical. Accordingly, the body 110 may be substantially thicker in a central portion relative to the perimeters of the upper and lower shells 120, 130. Such a tapered profile of the body 110 may ease the insertion of the disc replacement device 100 into a disc space. The tapered profile may also permit the disc replacement device 100 to at least partially extend into concave portions of adjacent vertebral bodies.

The upper and lower shells 120, 130 may be formed by extruding a profile comprising the upper and lower contacting surfaces 125, 135, by milling or otherwise machining the body 110 to define the upper and lower shells 120, 130, by casting, by injection molding, by combinations thereof, and/or by other manufacturing processes.

For example, one embodiment of a method of manufacturing the disc replacement device 100 may comprise providing the body 110 as formed by machining and/or extrusion and having a cross-sectional profile substantially conforming to the upper and lower contacting surfaces 125, 135. The cross-sectional profile may also include the fins 150, which may be formed by machining or extrusion. A footprint or top profile, best shown in FIG. 4, may then be defined from the body 110, such as by milling and/or otherwise machining. A plurality of through-holes 145 may be formed extending through the body 110, thereby defining the pillars 140. The through-holes 145 may be formed by drilling, boring, and/or otherwise removing material from the body 110. In one embodiment, the boring employed to form the through-holes 145 may result in pillars 140 that include fillets 147 proximate the upper and/or lower shells 120, 130. For example, in one embodiment, the through-holes 145 may have a substantially oval-shaped cross-section.

The pillars 140 may each be substantially similar in shape and/or cross-sectional profile. However, as in the illustrated embodiment, the pillars may also vary in shape and/or cross-sectional profile. For example, pillars 140 adjacent the perimeters of the upper and lower shells 120, 130 may have a first shape and a first cross-sectional profile, whereas pillars 140 interiorly offset from the perimeters of the upper and lower shells 120, 130 may have a second shape and a second cross-sectional profile. Moreover, while the illustrated embodiment includes eight pillars 140 adjacent the perimeters of the upper and lower shells 120, 130, and one pillar 140 interiorly offset from the perimeters, the scope of the present disclosure is not limited to any particular number of such perimeter and interior pillars 140.

The shape of the pillars 140 may also vary within an embodiment and among various embodiments. For example, the pillars 140 may have a substantially polygonal shape and/or a substantially polygonal cross-sectional profile. As shown in FIGS. 1-4, the pillars 140 may also have a substantially hour-glass shape. As shown in a subsequently illustrated embodiment, the pillars 140 may also be substantially diamond-shaped, possibly having a central aperture or through-hole. Although not shown, those skilled in the art will understand that the pillars 140 may take on other shapes, including a V-shape (including two members that are offset adjacent one of the upper and lower shells 120, 130 and coming together adjacent the other of the upper and lower shells 120, 130), a tetrahedron shape (a solid having polygonal faces), a cone shape (having substantially circular interfaces of different diameters with the upper and lower shells 120, 130), irregular shapes, and/or combinations thereof. Moreover, the pillars 140 may be bowed or skewed off-center, such that a compressive load applied to the pillars 140 may cause more bending motion than buckling.

The width and height of the pillars 140, relative to the orientations shown in FIGS. 1-4, may vary depending on the application, and are not limited by the scope of the present disclosure. In one embodiment, one or more of the pillars 140 may have a width that is substantially equivalent to an average thickness of one of the upper and lower shells 120, 130. One or more of the pillars 140 may also have a height-to-width ratio of less than about 5:1, wherein the height is measured between opposing, interior surfaces of the upper and lower shells 120, 130 and the width is a minimum width of the pillar 140. For example, the height-to-width ratio may range between about 1:1 and about 3:1. The pillars 140 may have a first cross-sectional area proximate the upper and/or lower shells 120, 130 and a second cross-sectional area distal from the upper and lower shells 120, 130, wherein the first cross-sectional area may be less than or greater than the second cross-section area.

As mentioned above, the disc replacement device 100 may also include fins 150. In the illustrated embodiment, the disc replacement device 100 includes two fins 150 extending from the upper contacting surface 125 and two fins 150 extending from the lower contacting surface 135. However, a fewer or greater number of fins 150 are also contemplated by the present disclosure. Moreover, the fins 150 extending from the upper contacting surface 125 may differ in number, shape, and/or composition from the fins 150 extending from the lower contacting surface 135.

The fins 150 may include an anchor 152 and a canted portion or tip 154, which is integral or otherwise coupled to the anchor 152. Although the anchor 152 and the tip 154 are shown as tetrahedrons or other prisms in the illustration, it is contemplated that they may each comprise other shapes. It is also contemplated that each of the anchor 34 and the tip 32 may comprise irregular shapes.

The anchor 152 may be integral to or otherwise coupled to one of the upper and lower shells 120, 130. In one embodiment, the anchor 152 comprises a composition that is more rigid or less-easily deformed relative to the tip 154. For example, the tip 154 may comprise a shape memory alloy and the anchor 152 may comprise stainless steel, titanium, polymers, carbon fiber or porous material. Thus, the tip 154 may be configured to deflect towards the upper and lower shells 120, 130 in response to a load or strain, whereas such deflection may be much less or not exist in the anchor 152. Consequently, the fins 150 may help retain the disc replacement device 100 in a disc space (not shown) and, thereby, resist rotation or translation within the disc space. In addition to the materials described above, the fins 150 may also comprise other biocompatible shape memory alloys, including Nitinol or copper-based alloys.

As with the pillars 140, the fins 150 may be deformed in response to a load or strain. For example, if such deformation occurs when the fins 150 are at a temperature less than a predetermined temperature, the deformation will remain. In one embodiment, this characteristic is utilized during installation of the disc replacement device 100 between two vertebral bodies. However, upon increasing the temperature of the fins 150 to a predetermined temperature, the fins 150 may return to their original shape. The predetermined temperature below which deformation remains may be a martensite temperature for the shape memory alloy. The predetermined temperature above which the fins 150 must be heated to return to their original shape may be the austenite temperature for a shape memory alloy. In one embodiment, the fins 150 may be deformed when at a temperature above the martensite temperature, such that the deformed fins 150 will substantially immediately attempt to return to their original shape.

The predetermined temperature(s) at which deformations of the fins 150 and the pillars 140 may be maintained or original shapes restored may be relative to the temperature range of a live human body in some embodiments. For example, the temperature at which the fins 150 and/or pillars 140 may be deformed and substantially maintain such deformation may be less than about 35° C., possibly at about 20° C. Similarly, the temperature at which the fins 150 and/or the pillars 140 may attempt to return to their original shape after deformation may range between about 35° C. and about 40° C. Moreover, the fins 150 and the pillars 140 may attempt to regain their initial shape even if the temperature at which they were deformed was not less than their martensite temperature.

Prior to insertion of the disc replacement device 100 into a disc space, the fins 150 may be cooled to a temperature, such as about 20° C., which is below its martensite temperature. At that point, the fins 150 may be deformed to a desirable position, such as by deflecting the fins 150 to be substantially parallel with the contact surfaces 125, 135, and the pillars 140 may be compressed in height, thereby facilitating the insertion of the disc replacement device 100 into the disc space.

The disc replacement device 100 may then be inserted into an intervertebral disc space as a single unit. Such insertion may utilize a delivery tube or sleeve into which the compressed disc replacement device 100 may be placed. The disc replacement device 100 may also be forcibly urged into the disc space by pushing on the trailing portion of upper and lower shells 120, 130, so that the leading portion of the disc replacement device 100 may be urged forward into the disc space.

In one embodiment, a portion of the disc space may be prepared prior to inserting the disc replacement device 100 to create a substantially cylindrical area in one or more of the adjacent vertebral bodies. Such cylindrical areas may receive the partially cylindrical portions of the upper and lower shells 120, 130. Thus, the contacting surfaces 125, 135 may substantially abut the prepared disc space. Similarly, slots or other shaped openings may be created in the adjacent vertebral bodies to host the fins 150.

In one embodiment, the prepared portion of the disc space may be limited to the area necessary to receive the disc replacement device 100, such that the rest of the disc space may remain unprepared. The unprepared portions of the disc space may engage the upper and lower shells 120, 130 to resist expulsion of the disc replacement device 100 from the disc space.

After the disc replacement device 100 is positioned in the disc space, the device 100 may be allowed to warm, such as to above the austenite temperature of the fins 150. Such warming may be in response to exposure to the temperature climate of the live human body, possibly ranging between about −35° C. and about 40° C. Accordingly, the fins 150 may attempt to return to their original shape, effectively wedging the disc replacement device 100 between adjacent vertebral bodies. In one embodiment, the disc replacement device 100 may be actively warmed by a heat source other than or in addition to the live human body. Such active heat sources may include a heat lamp, a hot air source, a visible or infrared light source, ohmic heating, and/or other sources.

Once installed, the pillars 140 may act as a shock absorber and/or load support, transmitting much of the compressive weight of the trunk and upper extremities between adjacent vertebral bodies. Accordingly, the pillars 140 may be configured to support at least the load and strain originally supported by the disc being replaced when the disc was healthy. Consequently, the pillars 140 may have an aspect ratio of less than about 5:1, as described above. While higher aspects ratios are within the scope of the present disclosure, such embodiments may insufficiently support the load and strain induced by motion of the spine. Moreover, because the pillars 140 may be configured to support at least the load and strain originally supported by the replaced disc, the disc replacement device 100 may include one or more interiorly offset pillars 140 because the perimeter pillars 140 may be insufficient to fully support such levels of load and strain in the absence of the interiorly offset pillars 140. Furthermore, the height of the pillars 140 combined with the thickness of the upper and lower shells 120, 130 (or the distance between the exterior surfaces 125, 135 of the shells 120, 130) may be approximately equal to the height of the disk replaced by the disc replacement device 100, such as when the disk was healthy.

During certain movements, such as bending or flexing, a heavy load or strain may be concentrated in one area of the disc replacement device 100. Responding to the heavy load, affected pillars 140 in that concentrated area may be reduced in height due to their superelastic nature. After the heavy load is removed, the pillars 140 may return to their original height.

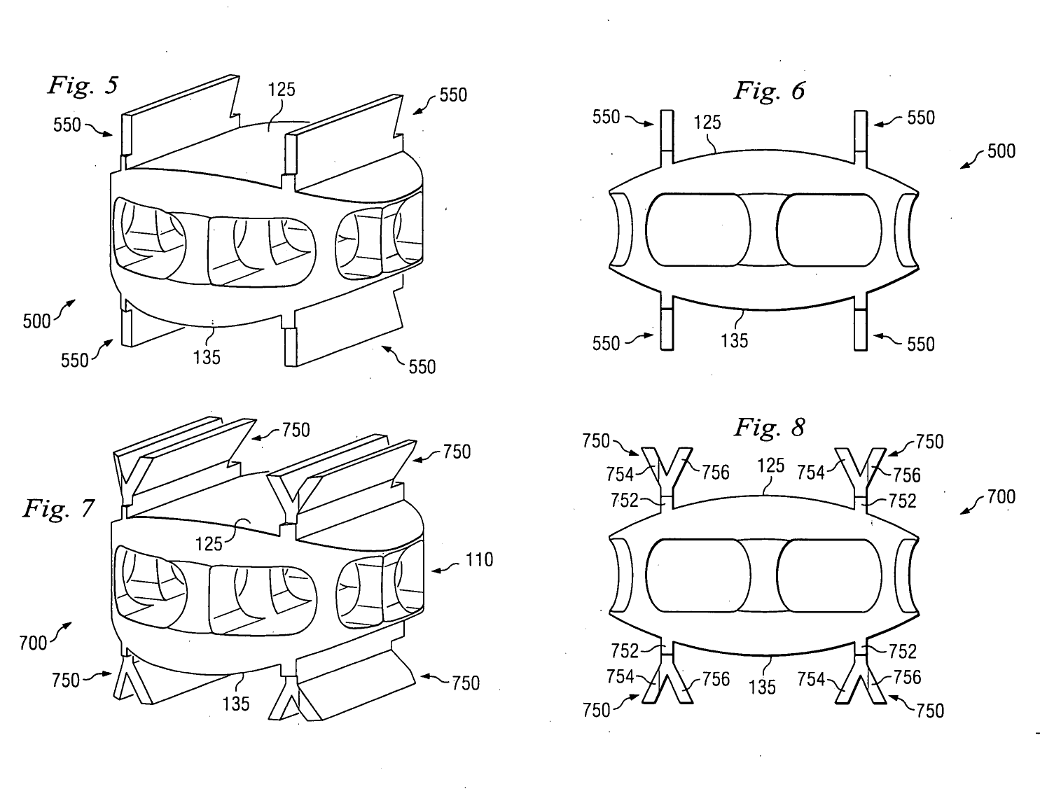

Referring to FIGS. 5 and 6 collectively, illustrated are various views of another embodiment of the disc replacement device 100 shown in FIGS. 1-4, herein designated by the reference numeral 500. The disc replacement device 500 is substantially similar to the disc replacement device 100 shown in FIGS. 1-4, with the exception that the device 500 includes fins 550 of a different configuration than the fins 150 shown in FIGS. 1-4.

That is, the fins 550 in the device 500 extend substantially perpendicular from the surfaces 125, 135, rather than being oriented at an acute angle relative to the surfaces 125, 135. Thus, load and strain applied to the fins 550 from adjacent vertebral bodies will cause the fins 550 to compress in height more than (or substantially instead of) deflecting towards the surfaces 125, 135.

Referring to FIGS. 7 and 8 collectively, illustrated are various views of another embodiment of the disc replacement device 100 shown in FIGS. 1-4, herein designated by the reference numeral 700. The disc replacement device 700 is substantially similar to the disc replacement device 100 shown in FIGS. 1-4, with the exception that the device 700 includes fins 750 of a different configuration than the fins 150 shown in FIGS. 1-4.

That is, the fins 750 in the device 700 have a multi-branched, Y-shaped profile extending away from the surfaces 125, 135, rather than having a single member oriented at an acute angle relative to the surfaces 125, 135. Thus, load and strain applied to the fins 750 from adjacent vertebral bodies may cause the fins 750 to compress in height and deflect towards the surfaces 125, 135.

The illustrated fins 750 comprise three portions 752, 754, 756. The fin portions 752 extend substantially perpendicular from the surfaces 125, 135. The portions 754, 756 extend from the portions 752 at an acute angle relative to surfaces 125, 135. The portions 754, 756 may be integral to or otherwise coupled (e.g., by adhesive or thermal bonding) to the portions 752. In one embodiment, the fins 750 substantially comprise one or more shape memory alloys, and may be integral to or otherwise be coupled to the upper and lower shells 120, 130. In another embodiment, the fin portions 752 may comprise material that does not exhibit superelastic properties, such as stainless steel, titanium, and other materials, and the portions 754, 756 may comprise one or more shape memory alloys.

As with the fins 150 shown in FIGS. 1-4 and the fins 550 shown in FIGS. 5 and 6, the fins 750 may substantially span the length of the surfaces 125, 135 in a primary direction of the disc replacement device 700. Of course, in other embodiments, the fins 750 may only span a portion of such length. Moreover, as with the fins 150 of FIGS. 1-4 and the fins 550 of FIGS. 5 and 6, the disc replacement device 700 may include a greater or fewer number of fins 750 than in the illustrated embodiment, and the number of fins 750 extending from the upper surface 125 may be different than the number of fins extending from the lower surface 135.

The present disclosure also contemplates fins having shapes and/or profiles other than those of fins 150, 550, 750 described above. For example, the fins may have a substantially U-shaped profile, or some other profile comprising one or more polygons, barbs, interruptions, acute angles, obtuse angles, etc. It is also contemplated that the fins may comprise regular shapes, as illustrated, or more irregular shapes. Moreover, single embodiments of disc replacement devices constructed according to aspects of the present disclosure may include fins of several different shapes. For example, one embodiment of a disc replacement device within the scope of the present disclosure may comprise a number of fins substantially similar to the fins 150 shown in FIGS. 1-4 as well as a number of fins substantially similar to the fins 550 shown in FIGS. 5 and 6 and/or the fins 750 shown in FIGS. 7 and 8.

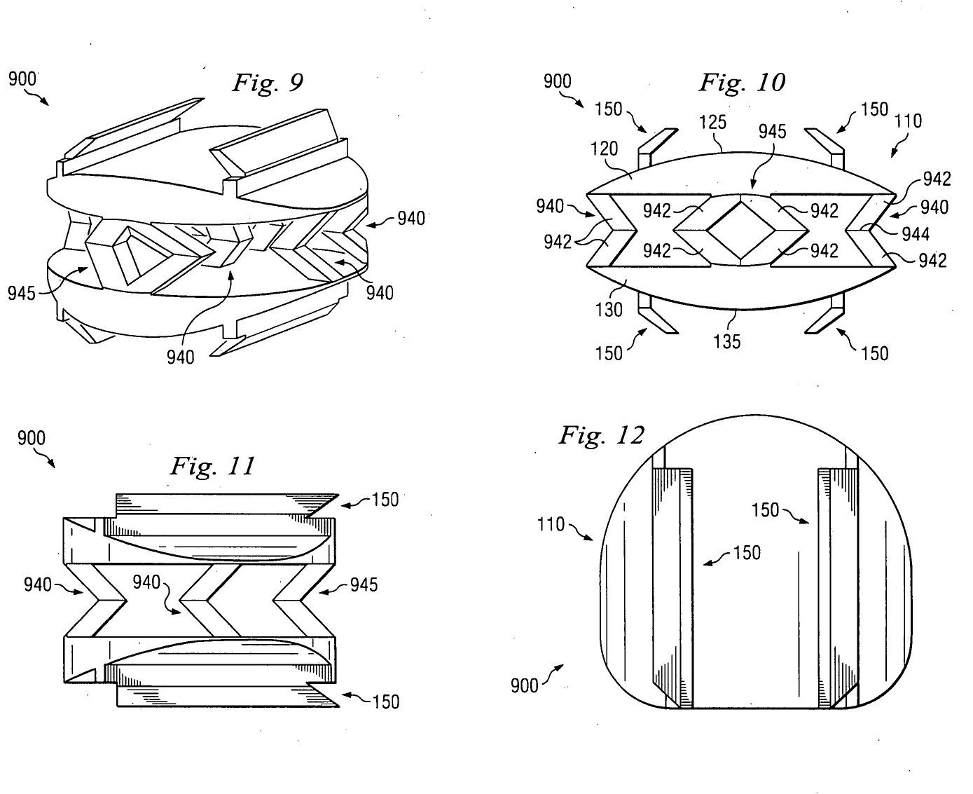

Referring to FIGS. 9-12 collectively, illustrated are various views of another embodiment of the disc replacement device 100 shown in FIGS. 1-4, herein designated by the reference numeral 900. The disc replacement device 900 is substantially similar to the disc replacement device 100 shown in FIGS. 1-4, with the exception that the device 900 includes pillars 940 of a different configuration than the pillars 140 shown in FIGS. 1-4.

That is, although otherwise substantially similar to the pillars 140 shown in FIGS. 1-4, the pillars 940 have a substantially V-shaped profile. The pillars 940 each include two members 942 extending from the upper and lower shells 120, 130 to an apex 944 approximately midway between the upper and lower shells 120, 130, wherein the apex 944 is interiorly or otherwise offset from a line extending between the interface points of the members 942 and the upper and lower shells 120, 130. The disc replacement device 900 may also include pillars 945 comprising four such members 942, the pillars 945 thereby having a substantially diamond-shaped profile with a central through-hole.

During movement of a spine in which the disc replacement device 900 is installed, the pillars 940, 945 may compress, such that the apex points 944 may travel in a direction substantially parallel to the interior surfaces of the upper and lower shells 120, 130, depending on the direction of offset of the apex points 944 in an unstrained condition. Of course, in embodiments in which the apex points 944 are not located approximately midway between the upper and lower shells 120, 130, such that the members 942 of a particular pillar 940, 945 may not have substantially equal lengths, the direction of travel of the apex points 944 in response to compression of the pillars 940, 945 may be at an acute angle relative to the interior surfaces of the upper and lower shells 120, 130.

Utilization of devices constructed according to aspects of the present disclosure will now be briefly described. It will be understood that access to the disc space, disc removal, and end plate preparation are known in the art and will be briefly described herein only. For example, procedures and instruments useable in a posterior approach to the disc space are disclosed in U.S. Pat. No. 6,241,729 (assigned to SDGI Holdings, Inc.), and a publication by Sofamor Danek© D1996 entitled “Surgical Technique using Bone Dowel Instrumentation for Posterior Approach”, each incorporated herein by reference in their entirety.

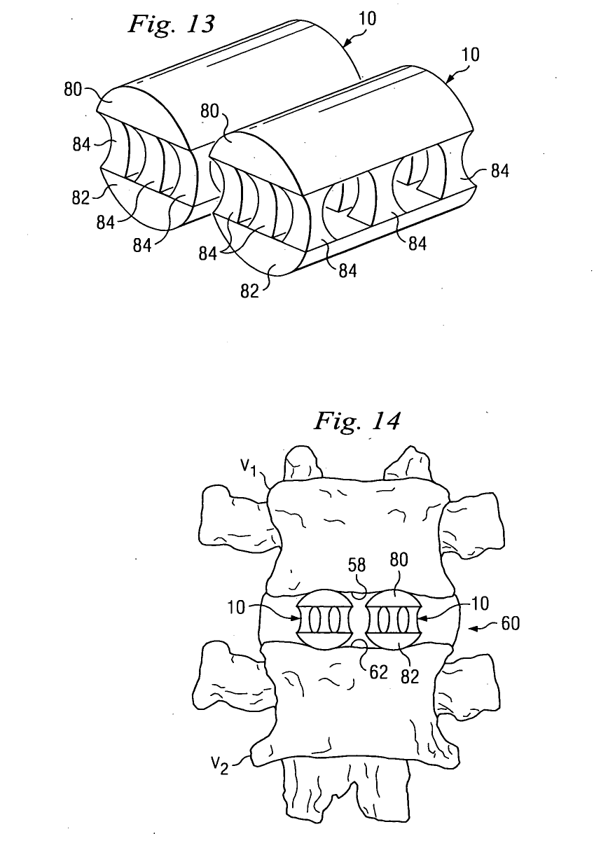

Referring to FIG. 13, illustrated is a perspective view of another embodiment of disc replacement devices 10 according to aspects of the present disclosure. The disc replacement devices 10 may be substantially similar to the devices 100, 500, 700, and/or 900 described above. The disc replacement devices 10 include upper and lower shells 80, 82, which may be substantially similar to the upper and lower shells discussed above. The upper and lower shells 80, 82 may form or contribute to a substantially circular, oval, ovoid, or cylindrical outer profile of the disc replacement devices 10. The disc replacement devices 10 also include pillars 84 coupled to and connecting the upper and lower shells 80, 82. The pillars 84 may be substantially similar to the pillars discussed above. As shown, the disc replacement devices 10 may include pillars 84 adjacent or proximate the perimeters of the shells 80, 82, as well as pillars 84 interiorly offset from the perimeters of the shells 80, 82. Although not shown in FIG. 13, the disc replacement devices 10 may also include fins extending from outer surfaces of the shells 80, 82, possibly extending along a substantial length of a primary dimension of each device 10.

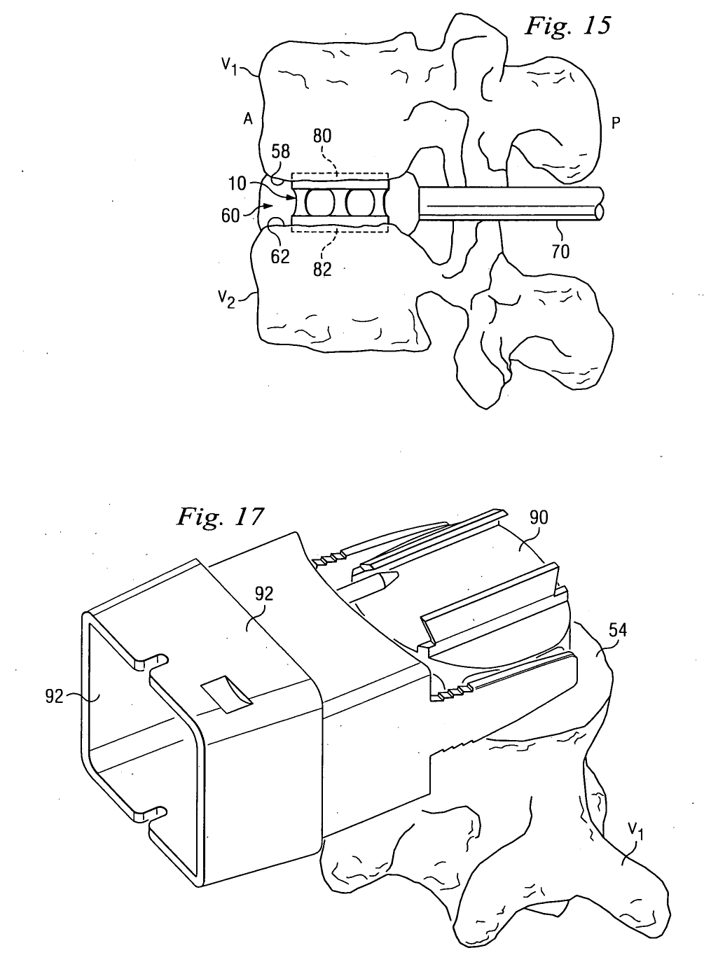

Referring to FIGS. 14 and 15, illustrated are front and side views, respectively, demonstrating one embodiment of the installation of the disc replacement devices 10 according to aspects of the present disclosure. A disc space 60 is shown in FIGS. 14 and 15 as located between an upper vertebral body V1 and a lower vertebral body V2. The anterior side of the vertebral bodies is indicated by the letter “A”, and their posterior side is indicated by the letter “P”. Two disc replacement devices 10 are positioned in the disc space 60, although other installation procedures may install only one disc replacement device 10 or more than the two disc replacement devices 10 shown in FIGS. 14 and 15.

Insertion preparation may be made by removing material from the disc space 60 and forming, by reaming, cutting, tapping, and/or other techniques, an arcuate portion 58 in the upper vertebral body V1. In procedures utilizing an insertion sleeve, a laminectomy may also be performed through the sleeve. Similarly, a corresponding and aligned arcuate portion 62 may be formed in the lower vertebral body V2. One or more disc replacement devices 10 may then be inserted into the disc space 60 such that the upper shell 80 contacts and/or engages the arcuate portion 58 and the lower shell 82 contacts and/or engages the arcuate portion 62. The disc replacement devices 10 may be installed one at a time, or more than one at a time. That is, an insertion tool employed during installation of the disc replacement devices 10 may have an internal cavity sized to receive more than one disc replacement device 10 (e.g., the insertion tool may be or comprise a double- or multi-barrel tube).

As shown in FIGS. 14-15, portions of bony material may remain anteriorly and/or posteriorly of the disc replacement device 10, such as to effectively countersink or otherwise secure the disc replacement device 10 in the disc space 60 and further prevent its expulsion from the disc space 60. A variety of procedures, including a posterior approach to the disc space 60, may be employed to implant the disc replacement devices 10 into the disc space 60. Furthermore, the insertion may be accomplished by utilizing a single-, double-, or multi-barrel tube or insertion sleeve 70 via pushing or threading the disc replacement devices 10 into position through the tube or insertion sleeve 70.

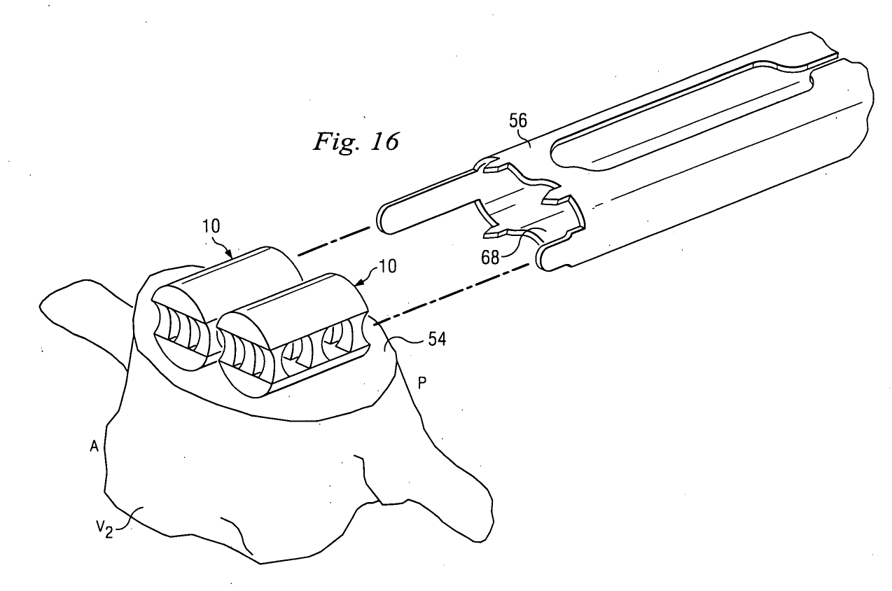

Referring to FIG. 16, illustrated is a perspective view demonstrating another embodiment of installation of one or more disc replacement devices 10 according to aspects of the present disclosure. A disc space 54 between vertebral bodies is configured for insertion of the disc replacement device 10 utilizing a double-barrel insertion sleeve 56. In operation, the insertion procedure may be performed by an anterior approach to the disc. space 54. Exemplary procedures and instruments which may be utilized in an anterior approach are disclosed in U.S. Pat. No. 6,428,541 (assigned to SDGI Holdings, Inc.), and a publication by Sofamor Danek© 1996 entitled “Surgical Technique using Bone Dowel Instrumentation for Anterior Approach”, each of which is incorporated herein by reference in their entirety.

An interior channel 68 of the insertion sleeve 56 and the disc replacement device 10 may be sized such that the disc replacement device 10 is maintained in a partially compressed condition during insertion, allowing its insertion into the disc space 54 in a reduced height state. It will be understood that the endplates of the vertebral body adjacent the disc space 54 are prepared to receive the disc replacement device 10 prior to its insertion. Techniques for shaping vertebral body endplates to conform them to the geometry of devices positioned in the disc space are well-known in the art and will not be further described herein. In one embodiment, the locations for the cylindrical shells of the disc replacement device 10 are prepared by reaming the disc space 54, such that the reamed disc space 54 permits the disc replacement device 10 to be countersunk or otherwise secured in the disc space 54 to prevent its expulsion from the disc space 54. After the disc replacement device 10 is inserted, fins on the device 10 (e.g., fins 150 shown in FIGS. 1-4) may also grab the vertebral bodies to maintain the disc replacement device 10 in the disc space 54. It is also contemplated that the disc replacement device 10 may be inserted by a lateral approach or other methods.

Referring to FIG. 17, illustrated is a perspective view demonstrating another embodiment of the installation of a disc replacement device 90 according to aspects of the present disclosure. The disc replacement device 90 is substantially similar to at least one of the disc replacement devices 100, 500, 700, and 900 described above. As in the illustrated embodiment, the insertion procedure may be performed by an anterior approach to the disc space 54. However, the insertion procedure may also be performed by a posterior approach, a lateral approach, an anterior oblique approach, and/or a posterior oblique approach.

A disc space 54 between vertebral bodies is configured for insertion of the disc replacement device 90 utilizing an insertion sleeve 92. The insertion sleeve may be substantially similar to the insertion sleeve 56 shown in FIG. 16. An interior channel 94 of the insertion sleeve 92 and the disc replacement device 90 may be sized such that the disc replacement device 90 is maintained in a partially compressed condition during insertion, allowing its insertion into the disc space 54 in a reduced height state. It will be understood that the endplates of the vertebral body adjacent the disc space 54 are prepared to receive the disc replacement device 90 prior to its insertion. Techniques for shaping vertebral body endplates to conform them to the geometry of devices positioned in the disc space are well-known in the art and will not be further described herein. In one embodiment, the location for the substantially cylindrical or other regular or irregular shaped profile of the disc replacement device 90 is prepared by reaming the disc space 54, such that the reamed disc space 54 permits the disc replacement device 90 to be countersunk or otherwise secured in the disc space 54 to prevent its expulsion from the disc space 54. Such preparation may also including preparing slots, lips, ledges, shoulders, or other features to be engaged by fins extending from the disc replacement device 90. Thus, after the disc replacement device 90 is inserted, the fins may also grab the vertebral bodies to maintain the disc replacement device 90 in the disc space 54.

The present disclosure contemplates providing a variety of sizes and shapes of pillars and fins for utilization with upper and lower shells to achieve the necessary adaptation of a disc replacement device into a disc space between vertebral bodies while taking into consideration a surgeon's access to a disc space. Even though the combinations have been disclosed herein as being applicable to a particular disc space, this is not a limitation on the use of such devices, and uses in other manners or other disc space is contemplated as being within the spirit of the present disclosure.

The foregoing has outlined features of several embodiments so that those skilled in the art may better understand the detailed description that follows. Those skilled in the art should appreciate that they may readily use the present disclosure as a basis for designing or modifying other processes and structures for carrying out the same purposes and/or achieving the same advantages of the embodiments introduced herein. Those skilled in the art should also realize that such equivalent constructions do not depart from the spirit and scope of the present disclosure, and that they may make various changes, substitutions and alterations herein without departing from the spirit and scope of the present disclosure.

1. A disc replacement device, comprising:
an upper shell;
a lower shell; and
a plurality of compressible pillars each coupled to and connecting the upper and lower shells and comprising a shape memory alloy, wherein at least one of the plurality of pillars is interiorly offset from perimeters of the upper and lower shells.

2. The disc replacement device of claim 1 wherein the plurality pillars each deform under a strain.

3. The disc replacement device of claim 1 wherein the plurality of pillars are each superelastic within a temperature range of a live human body.

4. The disc replacement device of claim 1 wherein the plurality of pillars are each superelastic at a temperature of above about 34° C.

5. The disc replacement device of claim 1 wherein the plurality of pillars each comprise substantially similar shape memory alloys.

6. The disc replacement device of claim 1 wherein a first one of the plurality of pillars comprises a first shape memory alloy and a second one of the plurality of pillars comprises a second shape memory alloy, wherein the first shape memory alloy differs from the second shape memory alloy.

7. The disc replacement device of claim 1 wherein at least one of the plurality of pillars comprises Nitinol.

8. The disc replacement device of claim 1 wherein at least one of the plurality of pillars comprises a copper-based alloy.

9. The disc replacement device of claim 1 wherein the plurality of pillars each comprise a substantially similar shape.

10. The disc replacement device of claim 1 wherein a first one of the plurality of pillars comprises a first shape and a second one of the plurality of pillars comprises a second shape, wherein the first shape differs from the second shape.

11. The disc replacement device of claim 1 wherein at least one of the plurality of pillars comprises a tetrahedron shape.

12. The disc replacement device of claim 1 wherein at least one of the plurality of pillars comprises an hour-glass shape.

13. The disc replacement device of claim 1 wherein at least one of the plurality of pillars comprises a V shape.

14. The disc replacement device of claim 1 wherein at least one of the plurality of pillars comprises a rectangular prism shape.

15. The disc replacement device of claim 1 wherein at least one of the plurality of pillars comprises a pyramid shape.

16. The disc replacement device of claim 1 wherein at least one of the plurality of pillars comprises a cone shape.

17. The disc replacement device of claim 1 wherein at least one of the plurality of pillars comprises an irregular shape.

18. The disc replacement device of claim 1 wherein at least one of the plurality of pillars comprises a substantially polygonal cross-sectional shape.

19. The disc replacement device of claim 1 wherein at least one of the plurality of pillars comprises a fillet adjacent one of the upper and lower shells.

20. The disc replacement device of claim 1 wherein at least one of the plurality of pillars comprises an upper fillet adjacent the upper shell and a lower fillet adjacent the lower shell.

21. The disc replacement device of claim 1 wherein at least one of the plurality of pillars comprises a first cross-sectional area proximate at least one of the upper and lower shells and a second cross-sectional area distal from the upper and lower shells, wherein the first cross-sectional area is substantially greater than the second cross-sectional area.

22. The disc replacement device of claim 1 wherein the plurality of pillars includes eight pillars proximate the perimeters of the upper and lower shells and one pillar interiorly offset from the perimeters of the upper and lower shells.

23. The disc replacement device of claim 1 wherein at least one of the plurality of pillars has a width substantially equivalent to an average thickness of at least one of the upper and lower shells.

24. The disc replacement device of claim 1 wherein at least one of the plurality of pillars has a height-to-width ratio of less than about 5:1, wherein the height is measured between opposing, interior surfaces of the upper and lower shells and the width is a minimum width of the pillar.

25. The disc replacement device of claim 1 wherein at least one of the plurality of pillars has a height-to-width ratio ranging between about 1:1 and about 3:1, wherein the height is measured between opposing, interior surfaces of the upper and lower shells and the width is a minimum width of the pillar.

26. The disc replacement device of claim 1 wherein, in a substantially non-deformed state, exterior surfaces of the upper and lower shells are separated by a distance substantially equivalent to a height of a disk replaced by the disc replacement device.

27. The disc replacement device of claim 1 further comprising one or more fins extending from at least one of the upper and lower shells.

28. The disc replacement device of claim 27 wherein the one or more fins comprise a shape memory alloy.

29. The disc replacement device of claim 28 wherein the one or more fins are originally shaped at a temperature that is within a temperature range of a live human body.

30. The disc replacement device of claim 28 wherein the one or more fins are originally shaped at a temperature that is above about 34° C.

31. The disc replacement device of claim 28 wherein the one or more fins are oriented at an acute angle relative to one of the upper and lower shells in a non-deformed state.

32. The disc replacement device of claim 28 wherein the one or more fins are substantially perpendicular to one of the upper and lower shells in a non-deformed state.

33. The disc replacement device of claim 28 wherein the one or more fins each include two branches, each branch oriented at an acute angle relative to the other branch and to one of the upper and lower shells in a non-deformed state.

34. The disc replacement device of claim 1 wherein an outer shape of the disc replacement device is substantially cylindrical.

35. The disc replacement device of claim 1 wherein a cross-sectional profile of the disc replacement device is substantially oval-shaped.

36. The disc replacement device of claim 1 wherein a cross-sectional profile of the disc replacement device is substantially circular.

37. A disc replacement device, comprising:
a shell; and
one or more fins located on an outer surface of the shell and comprising a shape memory alloy.

38. The disc replacement device of claim 37 wherein the one or more fins are originally shaped at a temperature that is within a temperature range of a live human body.

39. The disc replacement device of claim 37 wherein the one or more fins are originally shaped at a temperature that is above about 34° C.

40. The disc replacement device of claim 37 wherein the one or more fins comprises a plurality of fins each comprising a substantially similar shape memory alloy.

41. The disc replacement device of claim 37 wherein the one or more fins comprises a plurality of fins, including a first fin comprising a first shape memory alloy and a second fin comprising a second shape memory alloy, wherein the first shape memory alloy differs from the second shape memory alloy.

42. The disc replacement device of claim 37 wherein at least one of the one or more fins comprises Nitinol.

43. The disc replacement device of claim 37 wherein at least one of the one or more fins comprises a copper-based alloy.

44. The disc replacement device of claim 37 wherein a portion of at least one of the one or more fins comprises stainless steel.

45. The disc replacement device of claim 37 wherein the one or more fins comprises a plurality of fins each having a substantially similar shape.

46. The disc replacement device of claim 37 wherein the one or more fins comprises a plurality of fins, including a first fin having a first shape and a second fin having a second shape, wherein the first shape differs from the second shape.

47. The disc replacement device of claim 37 wherein at least one of the one or more fins is oriented at an acute angle relative to the shell outer surface.

48. The disc replacement device of claim 47 wherein the fin oriented at the acute angle is deflectable towards the shell outer surface in response to a load.

49. The disc replacement device of claim 48 wherein the deflectable fin is configured to return towards a pre-deflected orientation in response to exposure to a temperature that is within a temperature range of a live human body.

50. The disc replacement device of claim 37 wherein at least one of the one or more fins comprises:
an anchor portion oriented at a first angle relative to the shell outer surface; and
a tip portion oriented at a second angle relative to the shell outer surface, wherein the first and second angles are substantially different.

51. The disc replacement device of claim 37 wherein at least one of the one or more fins comprises:
an anchor portion comprising a first composition; and
a tip portion comprising a second composition, wherein the first and second compositions are substantially different.

52. The disc replacement device of claim 37 wherein at least one of the one or more fins comprises a Y shape.

53. The disc replacement device of claim 37 wherein at least one of the one or more fins comprises two branches, each branch oriented at an acute angle relative to the other branch and to the shell outer surface.

54. The disc replacement device of claim 37 wherein at least one of the one or more fins is substantially perpendicular to the shell outer surface.

55. The disc replacement device of claim 37 wherein at least one of the one or more fins substantially spans a primary dimension of the shell outer surface.

56. A disc replacement device comprising:
an upper shell;
one or more upper fins extending from an exterior surface of the upper shell, wherein at least one of the one or more upper fins comprises a first shape memory alloy;
a lower shell;
one or more lower fins extending from an exterior surface of the lower shell, wherein at least one of the one or more lower fins comprises a second shape memory alloy; and
one or more pillars connecting the upper shell and the lower shell, wherein at least one of the one or more pillars comprises a third shape memory alloy.

57. The disc replacement device of claim 56 wherein the first shape memory alloy is substantially similar to the second shape memory alloy.

58. The disc replacement device of claim 56 wherein the first shape memory alloy is substantially similar to the third shape memory alloy.

59. The disc replacement device of claim 56 wherein the second shape memory alloy is substantially similar to the third shape memory alloy.

60. A method of manufacturing a disc replacement device, comprising:
providing a shape memory alloy body; and
removing material from the body to form a plurality through-holes through the body, thereby defining, in the body, upper and lower shells and a plurality of integral pillars extending between the upper and lower shells.

61. The method of claim 60 wherein providing a shape memory alloy body includes providing a shape memory body having at least one fin integral to the body and extending from one of the exterior surfaces.

62. The method of claim 60 wherein providing the shape memory alloy body includes:
providing a shape memory alloy ingot; and
removing material from the ingot to form the exterior surfaces.

63. The method of claim 62 wherein removing material to form the exterior surfaces includes removing material to form at least one fin extending from one of the exterior surfaces.

64. The method of claim 60 wherein the plurality of through-holes each have substantially oval cross-sectional profile.

65. The method of claim 60 wherein removing material to form the plurality of through-holes comprises boring into the body.

66. A method of installing a disc replacement device, comprising:
providing a disc replacement device having upper and lower shells and a plurality of compressible pillars extending between the upper and lower shells, the plurality of pillars each comprising a shape memory alloy, at least one of the plurality of pillars interiorly offset from perimeters of the upper and lower shells; and
positioning the disc replacement device between adjacent vertebral bodies.

67. The method of claim 66 further comprising preparing a disc replacement region between the adjacent vertebral bodies prior to positioning the disc replacement device.

68. The method of claim 66 further comprising compacting the disc replacement device prior to positioning the disc replacement device between the adjacent vertebral bodies, wherein compacting the disc replacement device include compressing the pillars.

69. The method of claim 68 wherein compacting the disc replacement device comprises compressing the upper and lower shells towards each other, thereby compressing at least one of the plurality of pillars.

70. The method of claim 68 further comprising maintaining the disc replacement device below a predetermined temperature between compacting and positioning the disc replacement device.

71. The method of claim 68 wherein the disc replacement device includes at least one external fin extending from one of the upper and lower shells and compacting the disc replacement device comprises deflecting the at least one external fin towards one of the upper and lower shells.

72. The method of claim 68 further comprising installing the compacted disc replacement device in an insertion tool employed to position the disc replacement device between the adjacent vertebral bodies.

73. The method of claim 69 further comprising expanding the disc replacement device after positioning between the adjacent vertebral bodies.

74. The method of claim 73 wherein expanding the disc replacement device comprises heating the disc replacement device to at least a predetermined temperature.

75. The method of claim 74 wherein the predetermined temperature is within a temperature range of a live human body.
